SEQUENCE LISTING (3 sequences)

SEQ ID NO: 1 - protein, 1353 aa, Middle East Respiratory Syndrome (MERS)-coronavirus (CoV) (MERS-CoV 2)
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 2 - RNA, 4062 nt, Middle East Respiratory Syndrome (MERS)-coronavirus (CoV) (MERS-CoV 2)
augauacacucaguguuucuacugauguucuuguuaacaccuacagaaaguuacguugauguagggccagauucuguuaagucugcuuguauugagguugauauacaacagacuuucuuugauaaaacuuggccuaggccaauugauguuucuaaggcugacgguauuauauacccucaaggccguacauauucuaacauaacuaucacuuaucaaggucuuuuucccuaucagggagaccauggugauauguauguuuacucugcaggacaugcuacaggcacaacuccacaaaaguuguuuguagcuaacuauucucaggacgucaaacaguuugcuaauggguuugucguccguauaggagcagcugccaauuccacuggcacuguuauuauuagcccaucuaccagcgcuacuauacgaaaaauuuacccugcuuuuaugcuggguucuucaguugguaauuucucagaugguaaaaugggccgcuucuucaaucauacucuaguucuuuugcccgauggauguggcacuuuacuuagagcuuuuuauuguauucuagagccucgcucuggaaaucauuguccugcuggcaauuccuauacuucuuuugccacuuaucacacuccugcaacagauuguucugauggcaauuacaaucguaaugccagucugaacucuuuuaaggaguauuuuaauuuacguaacugcaccuuuauguacacuuauaacauuaccgaagaugagauuuuagagugguuuggcauuacacaaacugcucaagguguucaccucuucucaucucgguauguugauuuguacggcggcaauauguuucaauuugccaccuugccuguuuaugauacuauuaaguauuauucuaucauuccucacaguauucguucuauccaaagugauagaaaagcuugggcugccuucuacguauauaaacuucaaccguuaacuuuccuguuggauuuuucuguugaugguuauauacgcagagcuauagacugugguuuuaaugauuugucacaacuccacugcucauaugaauccuucgauguugaaucuggaguuuauucaguuucgucuuucgaagcaaaaccuucuggcucaguuguggaacaggcugaagguguugaaugugauuuuucaccucuucugucuggcacaccuccucagguuuauaauuucaagcguuugguuuuuaccaauugcaauuauaaucuuaccaaauugcuuucacuuuuuucugugaaugauuuuacuuguagucaaauaucuccagcagcaauugcuagcaacuguuauucuucacugauuuuggauuauuuuucauacccacuuaguaugaaauccgaucucaguguuaguucugcugguccaauaucccaguuuaauuauaaacaguccuuuucuaaucccacauguuugauuuuagcgacuguuccucauaaccuuacuacuauuacuaagccucuuaaguacagcuauauuaacaagugcucucgucuucuuucugaugaucguacugaaguaccucaguuagugaacgcuaaucaauacucacccuguguauccauugucccauccacugugugggaagacggugauuauuauaggaaacaacuaucuccacuugaaggugguggcuggcuuguugcuaguggcucaacuguugccaugacugagcaauuacagaugggcuuugguauuacaguucaauaugguacagacaccaauaguguuugccccaagcuugaauuugcuaaugacacaaaaauugccucucaauuaggcaauugcguggaauauucccucuaugguguuucgggccgugguguuuuucagaauugcacagcuguagguguucgacagcagcgcuuuguuuaugaugcguaccagaauuuaguuggcuauuauucugaugauggcaacuacuacuguuugcgugcuuguguuaguguuccuguuucugucaucuaugauaaagaaacuaaaacccacgcuacucuauuugguaguguugcaugugaacacauuuccucuaccaugucucaauacucccguucuacgcgaucaaugcuuaaacggcgagauucuacauaugguccccuucagacaccuguugguuguguccuaggacuuguuaauuccucuuuguucguagaggacugcaaguugccucuuggucaaucucucugugcucuuccugacacaccuaguacucucacaccucgcagugugcgcucuguuccaggugaaaugcgcuuggcauccauugcuuuuaaucauccuauucagguugaucaacuuaauaguaguuauuuuaaauuaaguauacccacuaauuuuuccuuuggugugacucaggaguacauucagacaaccauucagaaaguuacuguugauuguaaacaguacguuugcaaugguuuccagaagugugagcaauuacugcgcgaguauggccaguuuuguuccaaaauaaaccaggcucuccauggugccaauuuacgccaggaugauucuguacguaauuuguuugcgagcgugaaaagcucucaaucaucuccuaucauaccagguuuuggaggugacuuuaauuugacacuucuagaaccuguuucuauaucuacuggcagucguagugcacguagugcuauugaggauuugcuauuugacaaagucacuauagcugauccugguuauaugcaagguuacgaugauugcaugcagcaagguccagcaucagcucgugaucuuauuugugcucaauauguggcugguuacaaaguauuaccuccucuuauggauguuaauauggaagccgcguauaccucaucuuugcuuggcagcauagcagguguuggcuggacugcuggcuuauccuccuuugcugcuauuccauuugcacagaguaucuuuuauagguuaaacgguguuggcauuacucaacagguucuuucagagaaccaaaagcuuauugccaauaaguuuaaucaggcucugggagcuaugcaaacaggcuucacuacaacuaaugaagcuuuucacaagguucaggaugcugugaacaacaaugcacaggcucuauccaaauuagcuagcgagcuaucuaauacuuuuggugcuauuuccgccucuauuggagacaucauacaacgucuugauguucucgaacaggacgcccaaauagacagacuuauuaauggccguuugacaacacuaaaugcuuuuguugcacagcagcuuguucguuccgaaucagcugcucuuucggcucaauuggcuaaagauaaagucaaugagugugucaaggcacaauccaagcguucuggauuuugcggucaaggcacacauauaguguccuuuguuguaaaugccccuaauggccuuuacuucaugcauguugguuauuacccuagcaaccacauugagguuguuucugcuuauggucuuugcgaugcagcuaacccuacuaauuguauagccccuguuaauggcuacuuuauuaaaacuaauaacacuaggauuguugaugaguggucauauacuggcucguccuucuaugcaccugagcccaucaccucucuuaauacuaaguauguugcaccacaggugacauaccaaaacauuucuacuaaccucccuccuccucuucucggcaauuccaccgggauugacuuccaagaugaguuggaugaguuuuucaaaaauguuagcaccaguauaccuaauuuugguucucuaacacagauuaauacuacauuacucgaucuuaccuacgagauguugucucuucaacaaguuguuaaagcccuuaaugagucuuacauagaccuuaaagagcuuggcaauuauacuuauuacaacaaauggccgugguacauuuggcuugguuucauugcugggcuuguugccuuagcucuaugcgucuucuucauacugugcugcacugguuguggcacaaacuguaugggaaaacuuaaguguaaucguuguugugauagauacgaggaauacgaccucgagccgcauaagguucauguucacuaa

SEQ ID NO: 3 - DNA, 4062 bp, Artificial Sequence
  Synthetic DNA reverse transcription of Middle East Respiratory Syndrome (MERS)-coronavirus (CoV)  (MERS-CoV 2) RNA
atgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattatttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcctctaccatgtctcaatactcccgttctacgcgatcaatgcttaaacggcgagattctacatatggtccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacctcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcacaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttcggctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccatcacctctcttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttctctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaa

(19) United States
(12) Patent Application Publication
(10) Pub. No.: US 2022/0054628 A1
(43) Pub. Date: Feb. 24, 2022

(54) DEVELOPMENT OF AN EDIBLE VACCINE

(71) Applicant: King Abdulaziz University, Jeddah (SA)

(72) Inventors: Sayed Sartaj SOHRAB, Jeddah (SA); Esam Ibraheem Ahmed ASHAR, Jeddah (SA); Sherif Aly Abdelkhalek ELKAFRAWY, Jeddah (SA); Ayman Talaat Abbas Abdelhadi, Jeddah (SA); Edward P. RYBICKI, Jeddah (SA); Emmanuel Aubrey MARGOLIN, Jeddah (SA)

(21) Appl. No.: 16/997,313

(22) Filed: Aug. 19, 2020

(51) Int. Cl. A61K 39/215

(52) U.S. Cl. CPC A61K 39/215; A61K 2039/55561

(57) ABSTRACT

Plant-based, edible vaccines are provided. The vaccines are or are made from plants that are genetically engineered to express antigens of disease-causing microbes, for example, antigens of the MERS-CoV virus, such as the S1 subunit of the spike protein.

### BACKGROUND OF THE INVENTION

### Field of the Invention

This invention generally relates to plant-based, edible vaccines. In particular, the invention provides edible plants that are genetically engineered to express antigens of disease-causing microbes, for example, antigens of the Middle East Respiratory Syndrome (MERS)-coronavirus (CoV) (MERS-CoV) virus, and the use of the plants as vaccine vehicles.

### State of Technology

In the modern world, due to frequent travel and mixing of populations, the transmission of infectious diseases rapidly becomes a global problem. For example, the Middle East Respiratory Syndrome Coronavirus (MERS-CoV), which belongs to the family Coronaviridae, was first identified in Jeddah, Saudi Arabia in 2012 (1). Since then it has spread to 27 countries, with at least 2494 confirmed cases and 858 deaths. Thus, MERS-CoV has become a serious problem for human health globally. The virus originates from bats and transmission to humans occurs from camels which act as an intermediate host (2-3). The transmission of the virus from camels to humans has recently been confirmed based on full genome sequencing of both human and camel samples (4).

Currently, no licensed vaccine is available for MERS-CoV. However, the development of a protective vaccine is of great importance to prevent and control the spread of the virus and prevent future outbreaks. Several different types of vaccines utilizing various technologies are in the process of development, including orthopoxvirus vectors, recombinant adenoviruses, poxviruses, Modified Vaccinia Virus Ankara, measles virus, various viral-vector-based vaccines, nanoparticle-based vaccines, DNA-based vaccines, DNA prime/protein boost vaccines and sub-unit vaccines (5-6). Recently, a highly immunogenic, protective and safe adenovirus-based vaccine expressing MERS-CoV S1-CD40L fusion protein in a transgenic human DPP4 mouse model was developed and is under further evaluation (7). However, even if such vaccines are successful, they are often expensive to develop manufacture and administer. Due to high cost, storage concerns (e.g. refrigeration), transportation and requirements for trained medical personnel, an injectable vaccine cannot be easily accessible in developing countries. Additionally, various pathogenic organisms, bacterial and viral diseases can be easily transmitted by the re-use of needles. As a result, the World Health Organization has strongly recommended the development of new technologies for vaccine production.

There is an urgent need to provide effective, inexpensive and accessible vaccines and vaccine vehicles for infectious diseases such as MERS-CoV.

### SUMMARY OF THE INVENTION

Described herein are edible, plant-based vaccines that can be delivered orally. The vaccines are provided as plants or parts of plants or products made from plants, the plants having been genetically engineered to comprise one or more nucleic acids that encode one or more antigens of interest, such as antigens that elicit an immune response to one or more infectious agents. The encoded antigens are expressed within the plant or within at least one part of the plant and are generally delivered (administered) to a subject in need of a vaccine against the one or more infectious agents by oral consumption of the plant, or a part of the plant that contains the antigen(s), or a product made from the plant that contains the antigen(s). Such edible vaccines are relatively inexpensive to produce and are particularly suited for immunizing people e.g. in developing countries and/or remote regions where high production cost, transportation and the need for refrigeration otherwise hamper effective vaccination programs. The disclosure provides not only edible vaccines as new products but also a platform for edible vaccine production which can be further utilized to develop edible vaccines against many other diseases in multiple desired crops.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

It is an object of this invention to provide a transgenic plant, plant part or plant cell comprising a nucleic acid sequence encoding sub-unit 1 (S1) of the MERS-CoV spike glycoprotein (S1 MERS-CoV), and/or S1 MERS-CoV protein. In some aspects, the nucleic acid sequence is present in an expression vector. In some aspects, the transgenic plant, plant part or plant cell is a transgenic wheat plant, plant part or plant cell.

Also provided is a transgenic wheat plant, plant part or plant cell comprising sub-unit 1 (S1) of the MERS-CoV spike glycoprotein (S1 MERS-CoV) and/or a nucleic acid sequence encoding S1 MERS-CoV.

The disclosure also provides a method of producing a transgenic plant, comprising transforming a plant cell with a nucleic acid sequence encoding sub-unit 1 (S1) of the MERS-CoV spike glycoprotein (S1 MERS-CoV) and regenerating a plant from the transformed cell. In some aspects, the nucleic acid sequence is present in an expression vector. In additional aspects, the plant is wheat.

Also provided is a method of eliciting an immune response to MERS-CoV in a subject in need thereof, comprising providing to the subject an edible plant or a part of the edible plant, or a product made from the edible plant or the part of the edible plant, wherein the edible plant or the part of the edible plant is genetically engineered to contain and express a nucleic acid sequence encoding sub-unit 1 (S1) of the MERS-CoV spike glycoprotein (S1 MERS-CoV). In some aspects, the edible plant is wheat. In further aspects, the subject is a camel.

### DESCRIPTION OF THE DRAWINGS

FIG. 1. Exemplary amino acid sequence of an S1 protein of MERS-CoV (SEQ ID NO:1); see GenBank Protein Accession #AHE78108.1).

FIG. 2. Exemplary RNA sequence encoding the S1 protein of MERS-CoV (SEQ ID NO:2); see GenBank Accession #KF958702.1.

FIG. 3. Exemplary DNA sequence encoding the S1 protein of MERS-CoV (SEQ ID NO:3) based on a reverse translation of SEQ ID NO:2.

### DETAILED DESCRIPTION

Provided herein are genetically engineered (transgenic) plants comprising nucleotide sequences (e.g. DNA sequences) which encode one or more antigens from an infectious agent. When the plant is transformed with the nucleotide sequences, the one or more antigens are expressed (translated into protein) in at least one part of the plant, and when the plant (or parts of the plant comprising the translated antigens, or a product made from the plant of parts of the plant) is consumed by a subject, an immune response to the antigens is elicited in the subject. In some aspects, the genetically engineered plants thus serve as vehicles or vaccines for the delivery of the antigens to a subject. In some aspects, the developed vaccine is a plant-based edible vaccine for camels against MERS-CoV made by expressing sub-unit 1 (S1) of the MERS-CoV spike glycoprotein in a wheat crop. The vaccine is used to immunize camels against MERS-CoV, thereby breaking the chain of transmission to humans Thus, in some exemplary aspects, the methods described herein involve: PCR amplification and cloning of the MERS-CoV-S1 fragment; gene construct preparation; plant transformation and screening of transgenic wheat plants; and evaluation of immunogenicity and toxicity in mice and Camels.

The use of plant-based vaccines such as those described herein has many advantages. For example, no adjuvants are required to enhance immune responses; orally-introduced antigens elicit mucosal immunity; plants are easy to bulk produce onsite and can be transported and stored with low cost and without refrigeration since the antigens are stable in the plants; no injection is required, eliminating the need for specially trained medical person (no injection is required); ease of expression, separation and purification of proteinaceous antigens as needed; storage as seeds and oils and dried tissue without any refrigeration; no risk of contamination and disease spread, e.g. during manufacture; enhanced compliance, especially in children; and an increase the revenue and lowering of expenses. It is noted that edible vaccines are designed in such a way that, the expressed and produced proteins are not pathogenic. Because these vaccines are needle-free they have the added advantage of eliminating the waste and potential for dissemination of blood borne-infections associated with traditional vaccines.

### Definitions

Coronaviruses (CoV) are enveloped viruses with a positive (+) sense ssRNA genome (approximately 25.0 to 32.0 kb). CoV contain surface proteins which form “spikes”. The spikes are homotrimers of the S protein, which is composed of an S1 and S2 subunit. The S protein is a class I fusion protein which mediates receptor binding and membrane fusion between the virus and host cell. The S1 subunit forms the head of the spike and has the receptor binding domain (RBD), while the S2 subunit forms the stem which anchors the spike in the viral envelope and on protease activation enables fusion. The E and M protein are important in forming the viral envelope and maintaining its structural shape.

An “antigen” is a substance which induces an immune response in the body, especially the production of antibodies.

An “epitope” also known as antigenic determinant, is the part of an antigen that is recognized by the immune system, specifically by antibodies, B cells, or T cells. The epitope is the specific piece of the antigen to which an antibody binds. The part of an antibody that binds to the epitope is called a paratope. The epitopes of protein antigens may be conformational epitopes or linear epitopes.

“Expression” or “expressing” refers to production of a functional product, such as, the generation of an RNA transcript from an introduced construct, an endogenous DNA sequence, or a stably or transiently incorporated heterologous DNA sequence. A nucleotide encoding sequence may comprise intervening sequence (e.g., introns) or may lack such intervening non-translated sequences (e.g., as in cDNA). Expressed genes include those that are transcribed into mRNA and then translated into protein. The term may also refer to a polypeptide produced from an mRNA generated from any of the above DNA precursors. Thus, expression of a nucleic acid fragment, such as a gene or a promoter region of a gene, may refer to transcription of the nucleic acid fragment (e.g., transcription resulting in mRNA or other functional RNA) and/or translation of RNA into a precursor or mature protein (polypeptide), or both.

An “expression cassette” refers to a nucleic acid construct, which when introduced into a host cell, results in transcription and/or translation of a RNA or a polypeptide (or both), respectively. Expression cassettes are frequently housed within an “expression vector” or “expression construct” in order to introduce the nucleic acids encoded in the cassette into a host, e.g. by genetic engineering techniques.

The term “genome” as it applies to a plant cells encompasses not only chromosomal DNA found within the nucleus, but organelle DNA found within subcellular components (e.g., mitochondrial, plastid) of the cell. As used herein, the term “genome” refers to the nuclear genome unless indicated otherwise. However, expression in a plastid genome, e.g., a chloroplast genome, or targeting to a plastid genome such as a chloroplast via the use of a plastid targeting sequence, is also encompassed by the present disclosure.

The term “heterologous” refers to a nucleic acid fragment or protein that is foreign to its surroundings. In the context of a nucleic acid fragment, this is typically accomplished by introducing such fragment, derived from one source, into a different host (e.g. from a virus into a plant). Heterologous nucleic acid fragments, such as coding sequences that have been inserted into a host organism, are not normally found in the genetic complement of the host organism. A nucleic acid fragment that is heterologous with respect to an organism into which it has been inserted or transferred is sometimes referred to as a “transgene.” As used herein, the term “heterologous” also refers to a nucleic acid fragment derived from the same organism, but which is located in a different, e.g., non-native, location within the genome of this organism, within an expression vector, etc.

The term “homology” describes a mathematically based comparison of sequence similarities which is used to identify genes or proteins with similar functions or motifs. The nucleic acid and protein sequences of the present invention can be used as a “query sequence” to perform a search against public databases to, for example, identify other family members, related sequences, or homologs. The term “homologous” refers to the relationship between two nucleic acid sequence and/or proteins that possess a “common evolutionary origin”, including nucleic acids and/or proteins from superfamilies (e.g., the immunoglobulin superfamily) in the same species of animal, as well as homologous nucleic acids and/or proteins from different species of animal (for example, myosin light chain polypeptide, etc.; see Reeck et al., (1987) Cell, 50:667). Such proteins (and their encoding nucleic acids) may have sequence homology, as reflected by sequence similarity, whether in terms of percent identity or by the presence of specific residues or motifs and conserved positions. The methods disclosed herein contemplate the use of the presently disclosed nucleic and protein sequences, as well as sequences having sequence identity and/or similarity, and similar function.

“Host cell” means a cell which contains a vector (e.g. an expression vector) and supports the replication and/or expression of the vector. Host cells may be prokaryotic cells such as E. coli, or eukaryotic cells such as plant, yeast, insect, amphibian, or mammalian cells. The host cells are monocotyledonous or dicotyledonous plant cells.

The term “introduced” means providing a nucleic acid (e.g., an expression construct) or protein into a cell. “Introduced” includes reference to the incorporation of a nucleic acid into a eukaryotic or prokaryotic cell where the nucleic acid may be incorporated into the genome of the cell, and includes reference to either transient or stable provision of a nucleic acid or protein to the cell. “Introduced” thus includes reference to stable or transient transformation methods, as well as sexually crossing. Thus, “introduced” in the context of inserting a nucleic acid fragment (e.g., a recombinant DNA construct/expression construct) into a cell, can mean “transfection” or “transformation” or “transduction”, and includes reference to the incorporation of a nucleic acid fragment into a eukaryotic or prokaryotic cell where the nucleic acid fragment may be incorporated into the genome of the cell (e.g., chromosome, plasmid, plastid, or mitochondrial DNA), converted into an autonomous replicon, or transiently expressed (e.g., transfected mRNA).

The term “isolated” refers to a material such as a nucleic acid molecule, polypeptide, or small molecule that has been separated from the environment from which it was obtained. It can also mean altered from the natural state. For example, a polynucleotide or a polypeptide naturally present in a living animal is not “isolated” but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as “isolated polypeptides” or “isolated nucleic acid molecules”, etc., are polypeptides or nucleic acid molecules that have been purified, partially or substantially, from a recombinant host cell or from a native source.

As used herein, “nucleic acid” or “nucleotide sequence” means a polynucleotide (or oligonucleotide), including single or double-stranded polymers of deoxyribonucleotide or ribonucleotide bases, and unless otherwise indicated, encompasses naturally occurring and synthetic nucleotide analogues having the essential nature of natural nucleotides in that they hybridize to complementary single-stranded nucleic acids in a manner similar to naturally occurring nucleotides. Nucleic acids may also include fragments and modified nucleotide sequences. Nucleic acids disclosed herein can either be naturally occurring, for example genomic nucleic acids, or isolated, purified, non-genomic nucleic acids, including synthetically produced nucleic acid sequences such as those made by solid phase chemical oligonucleotide synthesis, enzymatic synthesis, or by recombinant methods, including for example, cDNA, codon-optimized sequences for efficient expression in different transgenic plants reflecting the pattern of codon usage in such plants, nucleotide sequences that differ from the nucleotide sequences disclosed herein due to the degeneracy of the genetic code but that still encode the protein(s) of interest disclosed herein, nucleotide sequences encoding the presently disclosed protein(s) comprising conservative (or non-conservative) amino acid substitutions that do not adversely affect their normal activity, PCR-amplified nucleotide sequences, and other non-genomic forms of nucleotide sequences familiar to those of ordinary skill in the art.

The protein-encoding nucleotide sequences, and promoter nucleotide sequences used to drive their expression, disclosed herein can be genomic or non-genomic nucleotide sequences. Non-genomic nucleotide protein-encoding sequences and promoters include, for example, naturally-occurring mRNA, synthetically produced mRNA, naturally-occurring DNA, or synthetically produced DNA. Synthetic nucleotide sequences can be produced by means well known in the art, including by chemical or enzymatic synthesis of oligonucleotides, and include, for example, cDNA, codon-optimized sequences for efficient expression in different transgenic plants and algae reflecting the pattern of codon usage in such organisms, variants containing conservative (or non-conservative) amino acid substitutions that do not adversely affect their normal activity, PCR-amplified nucleotide sequences, etc.

“Nucleic acid construct” or “construct” refers to an isolated polynucleotide which can be introduced into a host cell. This construct may comprise any combination of deoxyribonucleotides, ribonucleotides, and/or modified nucleotides. This construct may comprise an expression cassette that can be introduced into and expressed in a host cell.

“Operably linked” refers to a functional arrangement of elements. A first nucleic acid sequence is operably linked with a second nucleic acid sequence when the first nucleic acid sequence is placed in a functional relationship with the second nucleic acid sequence. For instance, a promoter is operably linked to a coding sequence if the promoter effects the transcription or expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

The terms “peptide”, “polypeptide”, and “protein” are used to refer to polymers of amino acid residues. These terms are specifically intended to cover naturally occurring biomolecules, as well as those that are recombinantly or synthetically produced, for example by solid phase synthesis.

The term “promoter” or “regulatory element” refers to a region or nucleic acid sequence located upstream or downstream from the start of transcription and which is involved in recognition and binding of RNA polymerase and/or other proteins to initiate transcription of RNA. Promoters may or may not be of plant origin. For example, promoters derived from plant viruses, such as the CaMV35S promoter, or from other organisms, can be used as discussed herein. Promoters useful in the present methods include, for example, constitutive, strong, weak, tissue-specific, cell-type specific, seed-specific, inducible, repressible, and developmentally regulated promoters.

The term “purified” refers to material such as a nucleic acid, a protein, or a small molecule, which is substantially or essentially free from components which normally accompany or interact with the material as found in its naturally occurring environment, and/or which may optionally comprise material not found within the purified material's natural environment. The latter may occur when the material of interest is expressed or synthesized in a non-native environment. Nucleic acids and proteins that have been isolated include nucleic acids and proteins purified by standard purification methods. The term also encompasses nucleic acids and proteins prepared by recombinant expression in a host cell as well as chemically synthesized nucleic acids.

“Recombinant” refers to a nucleotide sequence, peptide, polypeptide, or protein, expression of which is engineered or manipulated using standard recombinant methodology. This term applies to both the methods and the resulting products. As used herein, a “recombinant construct”, “expression construct”, “chimeric construct”, “construct” and “recombinant expression cassette” are used interchangeably herein.

As used herein, the phrase “sequence identity” or “sequence similarity” is the similarity between two (or more) nucleic acid sequences, or two (or more) amino acid sequences. Sequence identity is frequently measured as the percent of identical nucleotide or amino acid residues at corresponding positions in two or more sequences when the sequences are aligned to maximize sequence matching, i.e., taking into account gaps and insertions. One of ordinary skill in the art will appreciate that sequence identity ranges are provided for guidance only. It is entirely possible that nucleic acid sequences that do not show a high degree of sequence identity can nevertheless encode amino acid sequences having similar functional activity. It is understood that changes in nucleic acid sequence can be made using the degeneracy of the genetic code to produce multiple nucleic acid molecules that all encode substantially the same protein. Means for making this adjustment are well-known to those of skill in the art. When percentage of sequence identity is used in reference to amino acid sequences it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Sequence identity (or similarity) can be readily calculated by known methods, including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Computerized implementations of algorithms can be used (GAP, BESTFIT, PASTA, and TFASTA in the GCG Wisconsin Package, available from Accelrys, Inc., San Diego, Calif., United States of America), or by visual inspection. See generally, (Altschul, S. F. et al., J. Mol. Biol. 215: 403-410 (1990) and Altschul et al. Nucl. Acids Res. 25: 3389-3402 (1997)), as can the BLAST algorithm, which is described in (Altschul, S., et al., NCBI NLM NIH Bethesda, Md. 20894; & Altschul, S., et al., J. Mol. Biol. 215: 403-410 (1990).

A “transgenic” organism, such as a transgenic plant, is a host organism that has been stably or transiently genetically engineered to contain one or more heterologous nucleic acid sequences or fragments, including nucleotide coding sequences, expression cassettes, vectors, etc.

### Antigens

Antigens from a variety of infectious agents may be delivered to subjects in need thereof. In some aspects, an exemplary infectious agent is a coronavirus, examples of which include but are not limited to the Middle East Respiratory Syndrome Coronavirus (MERS-CoV).

When the infectious agent is MERS-CoV, the antigen that is genetically engineered for expression in a plant may be any protein that is part of the virus. However, in some aspects, an exemplary antigen is the S1 protein of MERS-CoV (S1-MERS-CoV). We will use only S1 gene. While the S1-MERS-CoV protein serves as the basis of some description provided herein, those of skill in the art will recognize that it is only one example, and that the teachings provided herein can be applied to a wide variety of antigens from a wide variety of infectious agents.

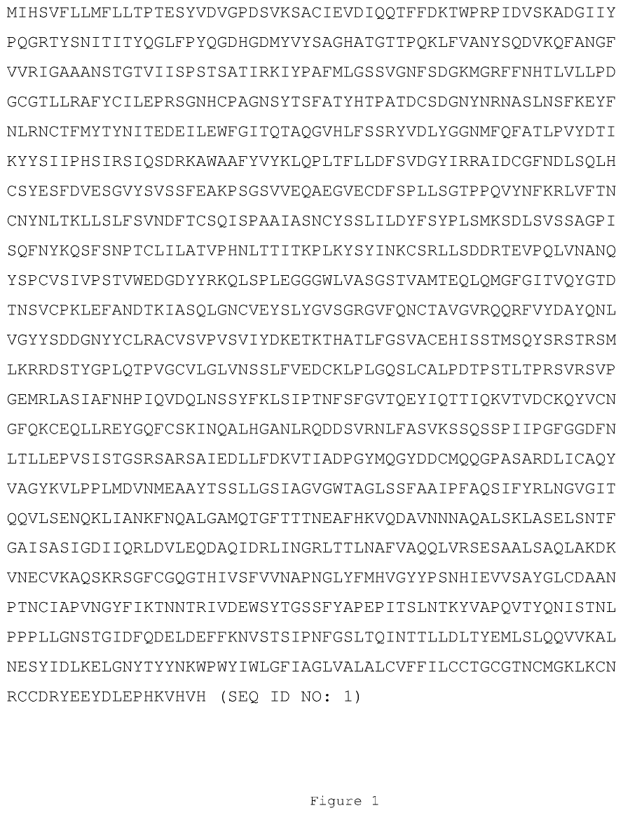

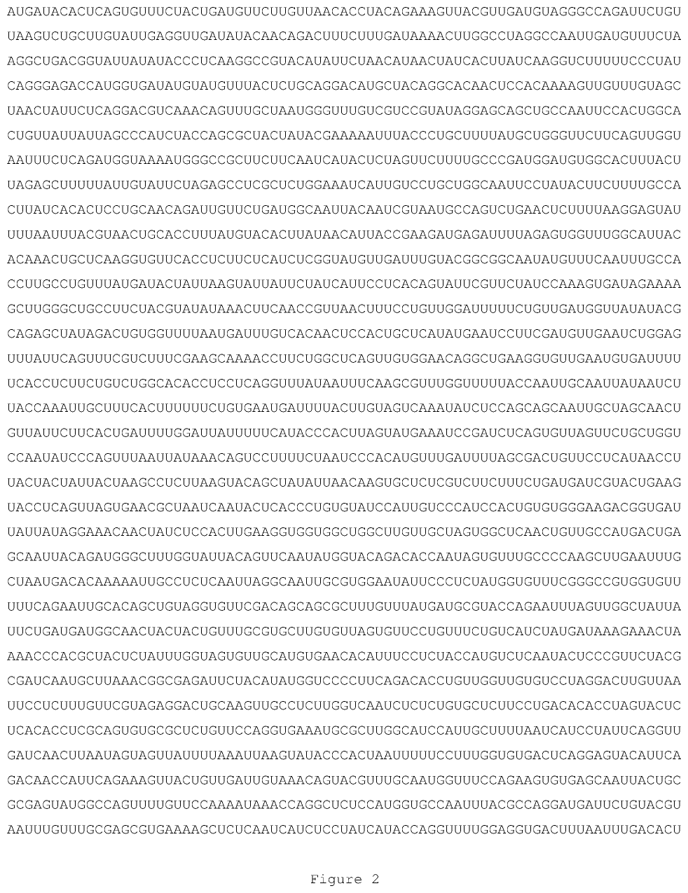

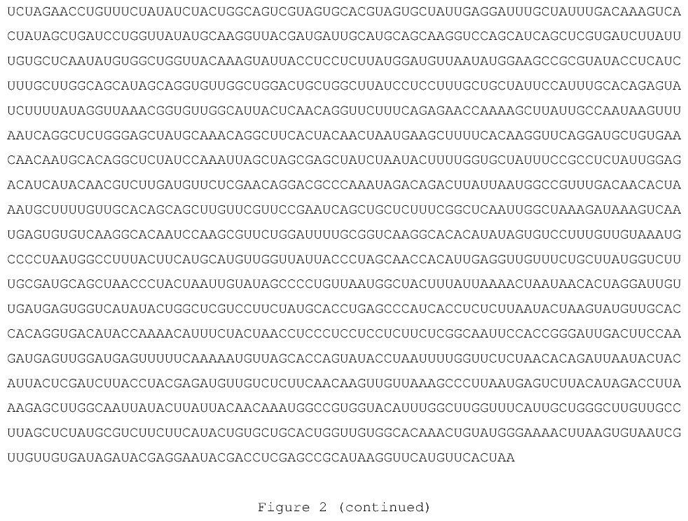

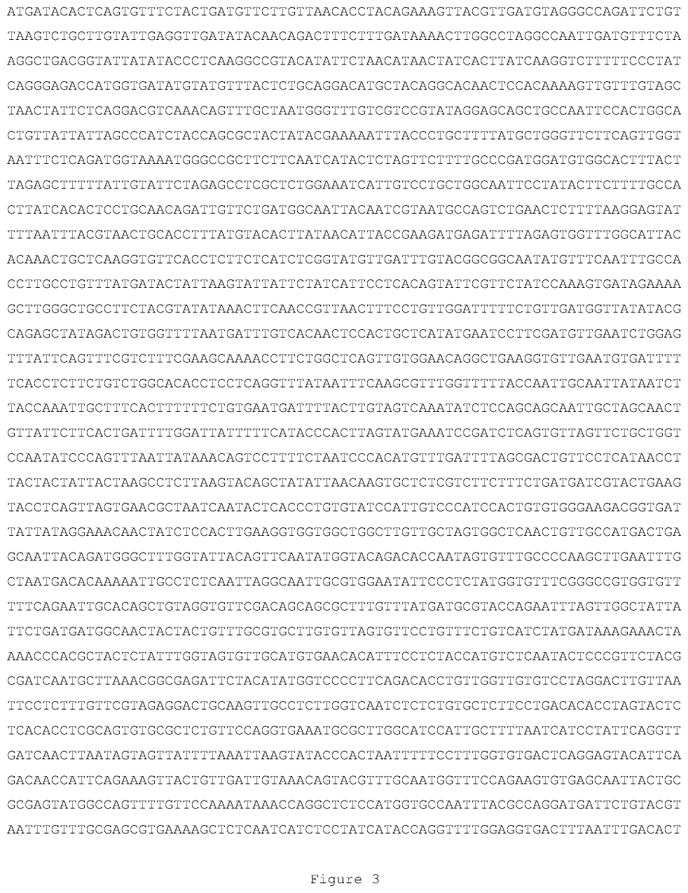

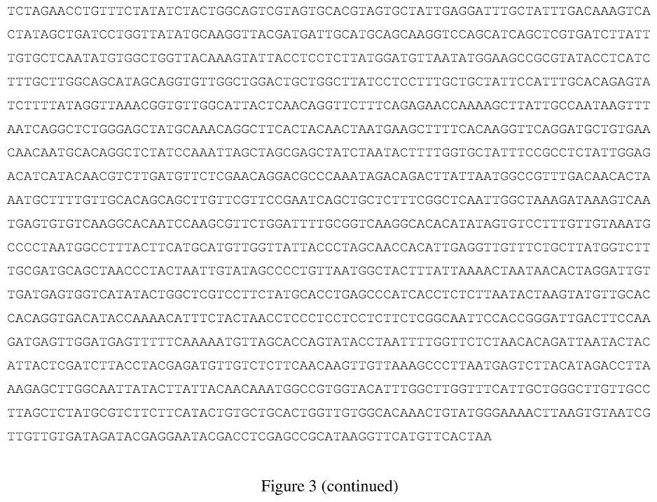

The amino acid sequence of an exemplary S1 protein of MERS-CoV is shown in FIG. 1. Those of skill in the art will understand that in nature, the S1 protein is encoded in the virus as + stand RNA, and an exemplary RNA gene sequence encoding the protein is depicted in FIG. 2. However, when used to genetically engineer a plant as described herein, DNA complementary to and or encoding the RNA is sometimes employed. An exemplary complementary DNA sequence that encodes the S1 protein and that can be inserted into a vector as described herein is shown in FIG. 3. While the invention may be implemented using the sequences disclosed herein, the constructs and methods disclosed herein encompass nucleic acid and protein sequences having sequence identity/sequence similarity at least about 75%, 80%, 85%, 90%, 91%, 92%, 93%, 94%, 95%, 96%, 97%, 98%, 99%, 100% to those specifically disclosed. In particular, natural variants, homologs or mutants of e.g. the S1-MERS-CoV protein may occur and be used as the basis for creating an artificial construct comprising a nucleic acid encoding the mutant protein, the construct then being used to transform a transgenic plant as described herein. A discussion of sequence identity/sequence similarity is provided in the “Definitions” section above.

In some aspects, the entire S1 sequence, which contains the receptor binding domain and is shown in SEQ ID NO: 1, is encoded as the “antigen”. In addition, a sequence such as the consensus S1 sequence shown in US patent application 20200222527 is used, as may other variants and versions thereof described in that application, the complete contents of which is herein incorporated by reference in entirety. However, those of skill in the art will recognize that short segments of the amino acid sequence of the protein may also be highly immunogenic and may function as antigenic determinants (epitopes). Any variation of the S1 spike protein may be used, as long as administration in an edible vaccine as described herein results in a beneficial immune response, such as a protective immune response, the elicitation of neutralizing antibodies, prevention or lessening of symptoms of infection, decreases death rate, etc. Generally, the “antigens” used herein comprise at least one such antigenic determinant.

In addition, certain truncated forms of the S1 protein may be used and still efficaciously elicit an immune response. For example, from about 1-5 amino acids (1, 2, 3, 4, or 5) may be removed from the amino- and/or carboxy terminus without having a deleterious effect on antigenicity.

Plants that can be Genetically Engineered

The terms “plant” or “plants” that can be used in the present methods broadly include the classes of higher and lower plants amenable to transformation techniques, including angiosperms (monocotyledonous and dicotyledonous plants), gymnosperms, and ferns. The term “plant” also includes plants which have been modified by breeding, mutagenesis, or genetic engineering (transgenic and non-transgenic plants). It includes plants of a variety of ploidy levels, including aneuploid, polyploid, diploid, haploid, and hemizygous. The plant may be in any form including suspension cultures, embryos, meristematic regions, callus tissue, gametophytes, sporophytes, pollen, microspores, whole plants, shoot vegetative organs/structures (e.g. leaves, stems and tubers), roots, flowers and floral organs/structures, seed (including embryo, endosperm, and seed coat) and fruit, plant tissue (e.g. vascular tissue, ground tissue, and the like) and cells, and progeny of same.

Aspects of the present disclosure also include parts of plants which can be selected from among a protoplast, a cell, a tissue, an organ, a cutting, an explant, a reproductive tissue, a vegetative tissue, biomass, an inflorescence, a flower, a sepal, a petal, a pistil, a stigma, a style, an ovary, an ovule, an embryo, a receptacle, a seed, a fruit, a stamen, a filament, an anther, a male or female gametophyte, a pollen grain, a meristem, a terminal bud, an axillary bud, a leaf, a stem, a root, a tuberous root, a rhizome, a tuber, a stolon, a corm, a bulb, an offset, a cell of said plant in culture, a tissue of said plant in culture, an organ of said plant in culture, a callus, propagation materials, germplasm, cuttings, divisions, and propagations.

Other aspects include progeny or derivatives of transgenic plants disclosed herein selected, for example, from among clones, hybrids, samples, seeds, and harvested material. Progeny can be asexually or sexually produced by methods well known in the art.

Plants to which the methods disclosed herein can be advantageously applied include but are not limited to both C3 and C4 plants, including “food crop” and “oilseed” plants. s is understood by those of skill in the art, the majority of plants and crop plants are C3 plants, referring to the fact that the first carbon compound produced during photosynthesis contains three carbon atoms. Under high temperature and light, however, oxygen has a high affinity for the photosynthetic enzyme Rubisco. Oxygen can bind to Rubisco instead of carbon dioxide, and through a process called photorespiration, oxygen reduces C3 plant photosynthetic efficiency and water use efficiency. In environments with high temperature and light, that tend to have soil moisture limitations, some plants evolved C4 photosynthesis. A unique leaf anatomy and biochemistry enables C4 plants to bind carbon dioxide when it enters the leaf and produces a 4-carbon compound that transfers and concentrates carbon dioxide in specific cells around the Rubisco enzyme, significantly improving the plant's photosynthetic and water use efficiency. As a result, in high light and temperature environments, C4 plants tend to be more productive than C3 plants. Examples of C4 plants include corn, sorghum, sugarcane, millet, and switchgrass. However, the C4 anatomical and biochemical adaptations require additional plant energy and resources than C3 photosynthesis, and so in cooler environments, C3 plants are typically more photosynthetically efficient and productive.

In some aspects, the plants that are genetically engineered as described herein are food crop plants. The term “food crop plant” refers to plants that are either directly edible, or which produce edible products, and that are customarily used to feed humans or animals either directly, or indirectly. Non-limiting examples of such plants include: 1. Cereal crops: wheat, rice, maize (corn), barley, oats, sorghum, rye, and millet; 2. Protein crops: peanuts, chickpeas, lentils, kidney beans, soybeans, lima beans; 3. Roots and tubers: potatoes, sweet potatoes, and cassavas; 4. Oil crops: soybeans, corn, canola, peanuts, palm, coconuts, safflower, cottonseed, sunflower, flax, olive, and safflower; 5. Sugar crops: sugar cane and sugar beets; 6. Fruit crops: bananas, oranges, apples, pears, breadfruit, pineapples, and cherries; 7. Vegetable crops and tubers: tomatoes, lettuce, carrots, melons, asparagus, etc. 8. Nuts: cashews, peanuts, walnuts, pistachio nuts, almonds; 9. Forage and turf grasses; 10. Forage legumes: alfalfa, clover; 11. Drug crops: coffee, cocoa, kola nut, poppy; 12. Spice and flavoring crops: vanilla, sage, thyme, anise, saffron, menthol, peppermint, spearmint, coriander. In some aspects, the plant is wheat.

The terms “oilseed plant” or “oil crop plant”, and the like, to which the present methods and compositions can also be applied, refer to plants that produce seeds or fruit with oil content in the range of from about 1 to 2%, e.g., wheat, to about 20%, e.g., soybeans, to over 40%, e.g., sunflowers and rapeseed (canola). These include major and minor oil crops, as well as wild plant species. Exemplary oil seed or oil crop plants useful in practicing the methods disclosed herein include, but are not limited to, plants of the genera Brassica (e.g., rapeseed/canola (Brassica napus; Brassica carinata; Brassica nigra; Brassica oleracea), Camelina, Miscanthus, and Jatropha; Jojoba (Simmondsia chinensis), coconut; cotton; peanut; rice; safflower; sesame; soybean; mustard; wheat; flax (linseed); sunflower; olive; corn; palm; palm kernel; sugarcane; castor bean; switchgrass; Borago officinalis; Echium plantagineum; Cuphea hookeriana; Cuphea pulcherrima; Cuphea lanceolata; Ricinus communis; Coriandrum sativum; Crepis alpina; Vernonia galamensis; Momordica charantia; and Crambe abyssinica.

In some aspects, the subject that is immunized is a camel. Since wheat is the preferred food for camels, in this aspect, the plant that is transformed may be wheat. Wheat has many advantageous properties, for example it has excellent biomass with less input and is heat stable.

### Production of Trans Genic Plants

In order to produce the transgenic plants described herein, early steps include identifying a target antigen to serve as the antigenic sequence to be expressed in the plant and obtaining a nucleic acid that encodes the antigen. Those of skill in the art are familiar with the processes that are involved, for example, the design of primers complementary to sequences that flank a genetic sequence that encodes the antigen in order to amplify the targeted genetic sequence, amplification of the sequence (e.g. by PCR), purifying the sequence and inserting it into a suitable vector. A suitable vector may be a vector used e.g. for maintenance and storage of the sequence for further manipulation such as sequencing; and/or a suitable vector for use in genetically engineering a plant. Examples of vectors include but are not limited to: plasmids, viral vectors, cosmids, and artificial chromosomes. Of these, the most commonly used vectors are plasmids. Common to all engineered vectors are an origin of replication, a multicloning site (into which the DNA encoding the antigen is inserted), and a selectable marker. Examples selectable or detectable markers include but are not limited to: antibiotic resistance markers (such as ampicillin, chloroamphenicol, tetracycline or kanamycin, etc.), etc. Vectors and constructs which encode the antigens disclosed herein are also encompassed.

In some aspects, the vector is an expression vector. An expression vector, otherwise known as an expression construct, is usually a plasmid or virus designed for gene expression in cells. The vector is used to introduce a specific gene into a target cell (e.g. a host cell) and can commandeer the cell's mechanism for protein synthesis to produce the protein encoded by the gene. The host cell may be a prokaryotic cell (e.g. a bacterial cell) or a plant cells. The disclosure encompasses vectors, expression vectors and host cells comprising at least one vector that comprises a (translatable, expressible) nucleotide sequence encoding at least one antigen of interest.

Typically a promoter is included in expression vectors. Suitable promoters for use in plants include but are not limited to the 35S CaMV promoter. In some aspects, the promoters are targeted promoters, e.g. promoters which direct expression of the antigens in one particular part of the plant, such as: tissue specific promotors (APRS, APRL, DLL, MXL, ESL [GenBank accession numbers CP02688.1 (location 6894692-6894019); CP02688.1 (location 6896568-6894019); CP002687.1 (location 9155519-9157550); CP002688.1 (location 15225206-15227733), CP002685.1 (location 673199-675267)], etc. If the plant that is transformed is wheat, promoters of special interest include but are not limited to the 35S CaMV promoter.

Conventional techniques of molecular biology, recombinant DNA technology, microbiology, and chemistry useful in practicing the methods of the present disclosure are described, for example, in Green and Sambrook (2012) Molecular Cloning: A Laboratory Manual, Fourth Edition, Cold Spring Harbor Laboratory Press; Ausubel et al. (2003 and periodic supplements) Current Protocols in Molecular Biology, John Wiley & Sons, New York, N.Y.; Amberg et al. (2005) Methods in Yeast Genetics: A Cold Spring Harbor Laboratory Course Manual, 2005 Edition, Cold Spring Harbor Laboratory Press; Roe et al. (1996) DNA Isolation and Sequencing: Essential Techniques, John Wiley & Sons; J. M. Polak and James O′D. McGee (1990) In Situ Hybridization: Principles and Practice; Oxford University Press; M. J. Gait (Editor) (1984) Oligonucleotide Synthesis: A Practical Approach, IRL Press; D. M. J. Lilley and J. E. Dahlberg (1992) Methods in Enzymology: DNA Structure Part A: Synthesis and Physical Analysis of DNA, Academic Press; and Lab Ref: A Handbook of Recipes, Reagents, and Other Reference Tools for Use at the Bench, Edited by Jane Roskams and Linda Rodgers (2002) Cold Spring Harbor Laboratory Press; Burgess and Deutscher (2009) Guide to Protein Purification, Second Edition (Methods in Enzymology, Vol. 463), Academic Press. Note also U.S. Pat. Nos. 10,696,977; 8,178,339; 8,119,365; 8,043,842; 8,039,243; 7,303,906; 6,989,265; US20120219994A1; and EP1483367B1. The entire contents of each of these texts and patent documents are herein incorporated by reference.

Introduction of heterologous nucleic acids into a host cell to create a transgenic cell is not limited to any particular mode of delivery, and includes, for example, microinjection, floral dip, adsorption, electroporation, vacuum infiltration, particle gun bombardment, whiskers-mediated transformation, liposome-mediated delivery, Agrobacterium-mediated transfer, the use of viral and retroviral vectors, CRISPR and TALEN technology, etc.

Once a plant is successfully transformed to contain and express genes encoding the S1 antigen(s), the cultivation and production (molecular pharming) of the pharmaceutical crops is generally performed in control production facilities such as greenhouses, or in plant tissue culture, or some suitable protected environment to prevent the general environmental release of the biopharmaceuticals.

### Compositions and Administration

The some aspects, the plant-based vaccine compositions disclosed herein are intended for oral consumption. Delivery may be direct, e.g. by consumption of a transgenic plant (or a portion of the plant) by the subject. Alternatively, the plant may be processed e.g. into pellets, powders, a liquid carrier, etc. which comprise the portions of the plant that comprise the antigens, and which are then ingested by the subject. Part of processing may include drying the plant/plant parts, followed e.g. by milling or grinding to form particles that can be made into various (pharmaceutical) compositions or formulations e.g. pellets, capsules, liquids, pastes, etc.

Such pharmaceutical compositions generally comprise at least one of the disclosed antigens, i.e. one or more than one (a plurality) of different compounds (e.g. 2 or more such as 2, 3, 4, 5, 6, 7, 8, 9, 10 or more) may be included in a single formulation. The compositions generally include plants or plant parts and, optionally, a pharmacologically suitable (physiologically compatible) carrier, which may be solid of liquid, and if liquid, may be aqueous or oil-based. The compositions are prepared as liquid solutions or suspensions, or as solid forms such as pellets, tablets, pills, powders and the like. Solid forms suitable for solution in, or suspension in, liquids prior to administration are also contemplated (e.g. lyophilized forms of the compounds), as are emulsified preparations. In some aspects, the processed plants or plant parts are mixed with excipients which are pharmaceutically acceptable and compatible with the active ingredients (antigens), e.g. pharmaceutically acceptable salts. Suitable excipients include, for example, water, saline, dextrose, glycerol, ethanol and the like, or combinations thereof. In addition, the composition may contain minor amounts of auxiliary substances such as wetting or emulsifying agents, pH buffering agents, preservatives, and the like. For oral forms of the compositions, various thickeners, flavorings, diluents, emulsifiers, dispersing aids or binders and the like are added. The composition of the present invention may contain any such additional ingredients so as to provide the composition in a form suitable for administration. The final amount of compound in the formulations varies but is generally from about 1-99%. Still other suitable formulations for use in the present invention are found, for example in Remington's Pharmaceutical Sciences, 22nd ed. (2012; eds. Allen, Adejarem Desselle and Felton).

Preparations can be standardized e.g. by sampling plants or portions of plants that are to be used in the compositions and using amounts that accord with the desired doses.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

“Pharmaceutically acceptable salts” refers to the relatively non-toxic, inorganic and organic acid addition salts, and base addition salts, of compounds of the present invention. These: salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-.beta.-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

While the compositions are typically administered orally, they may be administered by any suitable route including but not limited to: inoculation or injection, by absorption through epithelial or mucocutaneous linings (e.g., nasal, oral, vaginal, rectal, gastrointestinal mucosa, and the like).

In addition, the compositions may be administered in conjunction with other treatment modalities such as substances that boost the immune system, various chemotherapeutic agents, antibiotic agents, anti-inflammatory agents, and the like.

Subjects to whom the edible vaccines are administered are generally mammals. In some aspects, the subject is a camel. Any type of camel, wild or domesticated, may be a subject, including the one-humped dromedary, the two-humped Bactrian camel and the wild bactrian camel. In other aspects, the subject is a human Subjects may be of any age and may, for example, be juveniles, adults or “senior citizens”. Subjects may or may not have other underlying conditions. Subject may or may not already be infected with the infectious agent on which the antigens are based. In naïve subjects, the vaccine prevents disease; in infected patients, the vaccine may be used to treat the disease and/or to boost the immune system.

Administration may be a single event or “booster” administrations may be used, e.g. at intervals of a few weeks, a few months, a few years, etc. based on the final protocol that is established for the vaccine.

The amount of antigen that is administered to a subject varies from subject to subject e.g. according to age, gender, overall health, underlying conditions, etc. Those of skill in the art are best suited to determine the optimal amount.

In particular, the spread of MERS-CoV can be controlled by the immunization of camels because they are well known to be a natural reservoir for the spread of MERS-CoV infection to humans. The idea of camel immunization is a novel approach to break the chain of transmission. An edible vaccine provides a safe non-invasive, cost-effective and simple alternative approach for generating vaccines. Wheat is an important and preferable as well as desirable food for camels and the oral delivery of edible vaccine is easier and more feasible when the subjects are camels. The antigens in the edible vaccines are naturally protected after administration by bio-encapsulation and are thus stable in the intestinal tract. They can thus produce an immunogenic response in mucosa after absorbance in intestinal cell linings

### Immune Response

In some aspects, a suitable immune response involves the induction of neutralizing antibodies or antibodies with antiviral effector functions. These responses could confer sterilizing immunity by preventing the infection of susceptible cells, or by clearing virally infected cell, respectively Immunization could also elicit cellular immune responses against the antigen. Cytotoxic lymphocyte responses would be expected to eliminate virally infected cells. Cellular responses would also support the development of protective antibodies. Administration of the antigens described herein result in elicitation of an immune response in the subject to whom or to which the edible vaccine is administered. Preferably, the immune response is protective, i.e. prevents or at least lessens the development of at least one symptoms of MERS in a vaccinated subject that is later exposed to MERS-CoV, compared to an unvaccinated subject. While in some cases, administration may entirely prevent the development of symptoms, those of skill in the art will recognize that much benefit can accrue if the number or degree or time or persistence of symptoms are lessened, if not entirely eliminated. For example, one or more symptoms such as fever, cough, diarrhea, weakness, and even death, may be prevented or lessened by administration. The The immune response may include generation of neutralizing antibodies and/or a cellular response (such as one or preferably both of a T cell and B cell lymphocyte response), preferably neutralizing antibodies and at least a T cell response is elicited.

The mode of action of plant-based edible vaccine is that after ingestion, antigens are released from the vaccine via bio-encapsulation which protects them from gastric enzymes. The released proteins are absorbed by M cells in the intestinal wall and passed on to macrophages, antigen presenting cells and local lymphocyte populations which generate serum IgG, IgE and local IgA responses and memory cells which neutralize subsequent attacks by a real pathogen.

### Forms of the Plant-Based Vaccine

In some aspects, the plants or plant parts comprising antigens are consumed directly by the subject. In other aspect, the plants or plant parts comprising antigens are processed into an edible form. For example, they may be dried, milled, ground, etc. and formed into e.g. pellets, powders, etc. Accordingly, processed plant products, wherein the processed product comprises a detectable amount of an antigenic protein or antigenic fragment thereof, are also disclosed in this application. In certain embodiments, the processed product is, for example, one or more plant parts, plant biomass, oil, meal, animal feed, flour, pellets, flakes, bran, hulls, processed seed, and seed. In certain embodiments, the processed product is non-regenerable. The plant product can comprise commodity or other products of commerce derived from a transgenic plant or transgenic plant part, where the commodity or other products can be tracked through commerce by detecting nucleotide segments or expressed RNA or proteins that encode or comprise distinguishing portions of the protein. In some aspects, capsules are made from dried leaf tissue powder. For example, antigen containing wheat tissue powder can be converted into pellets or capsules. These products can advantageously be stored, usually without refrigeration, for e.g. weeks, months or even years, and the antigens remain stable and potent.

For standardization, different batches of plants or plant parts and/or powders made therefrom are blended to give known specific doses of antigen.

### Other Applications

Edible vaccines and the production platforms used to generate them in multiple desired crops provide solutions to treating and/or preventing various ailments and are advantageous compared to traditional vaccines, leading to safer and more effective immunization. For example, monoclonal antibodies are used in the treatment of arthritis and cancer and can be produced in transgenic plants with very low cost and rapidly.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

In this example, MERS-CoV sub-unit 1 (S1) protein is amplified and cloned into a plant transformation vector. For example, the gene construct is developed for the transformation of wheat. Wheat explants are transformed, e.g. by Agrobacterium and transgenic plants are screened and successfully transformed plants are selected for further evaluation, e.g. before harvesting of seeds. Oral immunization of mice and camels is performed using variable doses of pellets formed from transgenic wheat (leaves and/or tissue). The immunogenic and toxicity response is evaluated in mice using standard techniques. The best dose is selected for further evaluation of immunogenic responses in camels. Based on results from the camel study, the same technology is used for developing an edible vaccine for humans in one or more suitable crops.

### Methodology

Primer design: RNA isolation and amplification of sub-unit 1 (S1) is performed using specific primers based on sequence homology to GenBank sequences of MERS-CoV. The sub-unit 1 (S1) is PCR amplified using suitably designed primers.
Cloning and sequencing: The PCR amplified products are eluted, purified and ligated into a suitable cloning vector, such as a plasmid vector. Plasmid DNA is extracted and the insert size is confirmed by digestion and the sequence is confirmed by sequencing.
Gene construct development: Selected clones for which the size and sequence of the inert are confirmed are used to develop gene constructs by sub-cloning into a plant transformation vector. The resulting gene construct is mobilized into Agrobacterium for transient expression in tobacco and wheat plants.
Transformation of wheat explants: A suitable variety of wheat is transformed using the gene construct comprising the MERS-CoV sub-unit 1 (S1) gene. The transformed wheat plant cells are selected using appropriate antibiotic selection and further regenerated into mature plants.
Screening and harvesting of transgenic plants: The transgenic plants are screened for the presence and integrity of the MERS-CoV S1 protein, and the expression of the S1 protein/gm of plant tissue is analyzed by using standard techniques. The transgenic wheat seeds will be collected for further germination and toxicity and immunogenicity studies.
Evaluation of Immunogenicity and toxicity: Before starting an animal study, ethical approval is provided by the ethical committee of King Abdulaziz University Hospital.
Mouse immunization assay: Transgenic and non-transgenic wheat seeds are germinated and the resulting transgenic plants are used for immunization and toxicity evaluation after oral delivery. S-1 protein expressed in wheat leaf tissue is fed to the mice and the protein is released and absorbed through cells of the intestinal lining. The immune system of the mice identify the S-1 protein, and/or antigens and antigenic determinants present in the protein, as immunogens. In all experiments, 8 week-old Balb/c mice are used. Each group will have 10 animals and three doses of antigen will be administered on days 1, 7 and 14. Groups 1, 2 and 3 animals are orally administered by gavage using 100 μg, 200 μg and 500 μg of expressed protein in the form of compressed pellets made from transgenic wheat leaves. Group 4: receives a pellet from non-transgenic wheat leaves as a negative control. Group 5: receives purified SI protein as a positive control. On day 20, the mice are bled from retro-orbital plexus under deep anesthesia and the blood samples are stored at −20° C. The frequency of oral immunization are increased (or not) based on requirements determined after analysis of initial results. The immunogenic response is evaluated by a virus neutralization assay and ELISA.

Toxicity study: In all experiments 8 week-old Balb/c mice are used. Acute toxicity studies are performed on both male and female mice. Mice are tested with variable doses by oral gavage using compressed pellets made from transgenic and non-transgenic wheat leaves. After 14 days, the mice are sacrificed and their livers, kidneys, lungs, and hearts are examined macroscopically and histopathologically for any changes in weight or shape or pathology. Biochemical assessments for blood sugar, kidney function (urea and creatinine) and liver function (ALT, AST, albumin, bilirubin) are performed.

Camel Immunization assay: This study is conducted on 25-50 camels aged 7 to 10 years fasted overnight. Variable as well as higher doses based on the results obtained from previous mouse studies are used. The number, frequency and amount of antigen per dose is higher than the doses used in mice study. The camels are divided into 5 groups. The group 1, 2 and 3 camels are fed orally using variable doses of S1 protein expressed in transgenic wheat in the form of compressed pellets. Group 4 will receive purified S1 protein and an inactivated MERS-CoV intramuscularly as positive control. Group 5, animals are fed 1000 gm of non-transgenic wheat (negative control) Animals are boosted with the same doses after 7 and 14 days. Samples are collected (e.g. serum and/or nasal swabs) and stored at −20° C. Serum samples are tested for S1-MERS-CoV neutralizing antibody using ELISA techniques and serum neutralization assays.

It has been reported that antibody stability and protection in camels with a virus infection varies over time. Based on recently published information, variation in the stability of antibodies in camels always occurs and enables the virus to infect or re-infect camels. Thus, both seronegative (naïve) and seropositive camels are used in this study to examine the possibility of generating an enhanced immune response. Repeated immunizations are given to seropositive camels to protect from virus infection and by this protection, the chain is broken and the chances of virus spread from camels to human is reduced.

Challenge studies: For camels used in this study, the immunization, collection of samples (nasal swab and serum) is performed until a predetermined level of immune response indicators (antibodies, T cells, etc.) is achieved. Thereafter, camels are exposed to the virus and the level of protection and/or the level of infectious virions shed by the animal is measured by observing clinical symptoms and/or quantifying the viral load

While the invention has been described in terms of its preferred embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

1.-7. (canceled)

8. A method of eliciting a mucosal immune response to Middle East Respiratory Syndrome coronavirus (MERS-CoV) in a subject in need thereof, said method comprising
feeding the subject an edible wheat plant or a part of the edible wheat plant selected from the group consisting of a protoplast, a cell, a tissue, an organ, a cutting, an explant, a vegetative tissue, biomass, an inflorescence, a flower, a sepal, a petal, a pistil, a stigma, a style, an ovary, an ovule, an embryo, a receptacle, a seed, a stamen, an anther, a male or female gametophyte, a pollen grain, a meristem, a leaf, a stem and a root, wherein the edible wheat plant or the part of the edible wheat plant is genetically engineered to contain and express a nucleic acid sequence encoding sub-unit 1 of the MERS-CoV spike glycoprotein (S1 MERS-CoV),
wherein the edible wheat plant or the part of the edible wheat plant is orally ingested and the S1 MERS-CoV is released from bioencapsulation in an intestine and taken up into M cells and induces the mucosal immune response; and
wherein the subject is a camel.

9-10. (canceled)

11. A method of controlling spread of Middle East Respiratory Syndrome coronavirus (MERS-CoV) infection to humans by immunization of camels in an environment which includes humans and camels in proximity, comprising the steps of
feeding an edible wheat plant or a part of the edible wheat plant selected from the group consisting of a protoplast, a cell, a tissue, an organ, a cutting, an explant, a vegetative tissue, biomass, an inflorescence, a flower, a sepal, a petal, a pistil, a stigma, a style, an ovary, an ovule, an embryo, a receptacle, a seed, a stamen, an anther, a male or female gametophyte, a pollen grain, a meristem, a leaf, a stem and a root expressing sub-unit 1 of the MERS-CoV spike glycoprotein (S1 MERS-CoV) to the camels;
inducing a mucosal immunogenic response in the camels after release from bioencapsulation in the camels' intestines and absorbance in intestinal M cells in the absence of an adjuvant, and
breaking a chain of transmission of MERS-CoV from the camels to humans.

12. A method of eliciting a mucosal immune response to Middle East Respiratory Syndrome coronavirus (MERS-CoV) in a camel in need thereof, said method comprising
feeding the camel an edible vaccine consisting of a wheat plant or at least one part of the wheat plant selected from the group consisting of a protoplast, a cell, a tissue, an organ, a cutting, an explant, a vegetative tissue, biomass, an inflorescence, a flower, a sepal, a petal, a pistil, a stigma, a style, an ovary, an ovule, an embryo, a receptacle, a seed, a stamen, an anther, a male or female gametophyte, a pollen grain, a meristem, a leaf, a stem and a root, wherein the wheat plant or the part of the wheat plant is genetically engineered to contain and express a nucleic acid sequence encoding sub-unit 1 of the MERS-CoV spike glycoprotein (S1 MERS-CoV),
wherein the vaccine is orally ingested and the S1 MERS-CoV is released from bioencapsulation in the camel's intestine and taken up into M cells and induces the mucosal immune response.
